(19) United States
(12) Patent Application Publication
(10) Pub. No.: US 2021/0134453 A1
(43) Pub. Date: May 6, 2021

(54) APPARATUS AND METHOD FOR ESTIMATING BIO-INFORMATION

(71) Applicant: SAMSUNG ELECTRONICS CO., LTD., Suwon-si (KR)

(72) Inventors: Sang Kon BAE, Seongnam-si (KR); So Young Lee, Daejeon (KR)

(73) Assignee: SAMSUNG ELECTRONICS CO., LTD., Suwon-si (KR)

(21) Appl. No.: 16/832,467

(22) Filed: Mar. 27, 2020

(30) Foreign Application Priority Data
Oct. 30, 2019 (KR) 10-2019-0136304

(51) Int. Cl. G16H 40/63

(52) U.S. Cl. CPC G16H 40/63

(57) ABSTRACT

An apparatus for estimating bio-information is provided. The apparatus includes: a sensor configured to obtain a calibration data set and an estimation data set from an object; and a processor configured to set outlier removal criteria and generate an estimation model based on the calibration data set, and estimate bio-information based on the estimation data set, the outlier removal criteria, and the estimation model.

### CROSS-REFERENCE TO RELATED APPLICATION(S)

This application claims priority from Korean Patent Application No. 10-2019-0136304, filed on Oct. 30, 2019, in the Korean Intellectual Property Office, the entire disclosure of which is incorporated herein by reference for all purposes.

### BACKGROUND

### 1. Field

Apparatuses and methods consistent with example embodiments relate to non-invasively estimating bio-information such as blood glucose.

### 2. Description of Related Art

Diabetes is a chronic disease that causes various complications and can be hardly cured, such that people with diabetes are advised to check their blood glucose regularly to prevent complications. In particular, when insulin is administered to control blood glucose, the blood glucose levels have to be closely monitored to avoid hypoglycemia and control insulin dosage. An invasive method of finger pricking is generally used to measure blood glucose levels. However, while the invasive method may provide high reliability in measurement, it may cause pain and inconvenience as well as an increased risk of disease infections due to the use of injection. Recently, research has been conducted on methods of non-invasively measuring blood glucose by using a spectrometer without blood sampling.

### SUMMARY

According to an aspect of an example embodiment, there is provided an apparatus for estimating bio-information, the apparatus including: a sensor configured to obtain a calibration data set and an estimation data set from an object; and a processor configured to set outlier removal criteria and generate an estimation model based on the calibration data set, and estimate bio-information based on the estimation data set, the outlier removal criteria, and the estimation model.

The sensor may include: one or more light sources configured to emit light of a plurality of wavelengths onto the object; and a detector configured to detect the light when the light is reflected or scattered from the object.

The processor may remove outlier data from the calibration data set based on the outlier removal criteria, and may generate the estimation model based on the calibration data set, from which the outlier data is removed.

The processor may set the outlier removal criteria for each wavelength of the calibration data set.

The processor may detect an outlier for each wavelength from the calibration data set by applying a pre-defined outlier detection technique, and may set the outlier removal criteria for each wavelength based on the detected outlier.

The processor may remove the outlier data from the estimation data set based on the outlier removal criteria, and may estimate bio-information based on the estimation data set, from the outlier data is removed, and the estimation model.

The processor may detect the outlier data from the estimation data set based on the outlier removal criteria, and upon detecting the outlier data, the processor may control the sensor to re-obtain corresponding estimation data.

By driving a light source of a wavelength, at which the outlier is detected, the sensor may re-obtain data for the wavelength.

The sensor may include a plurality of light sources configured to emit light of a plurality of wavelengths to the object, and the processor may be further configured to drive all of the plurality of light sources to re-obtain estimation data for all of the plurality of wavelengths.

The processor may detect an outlier from the estimation data set, and may determine whether to perform calibration based on the detected outlier.

The apparatus for estimating bio-information may further include an output interface configured to output at least one of the outlier removal criteria, the estimation model, and the bio-information.

The apparatus for estimating bio-information may further include a communication interface configured to transmit at least one of the outlier removal criteria, the estimation model, and the bio-information to an external device.

The bio-information may include at least one of blood glucose, cholesterol, triglyceride, protein, and uric acid.

According to an aspect of another example embodiment, there is provided a method of estimating bio-information, the method including: obtaining calibration data; setting outlier removal criteria based on the calibration data; generating an estimation model based on the calibration data; obtaining estimation data; and estimating bio-information based on the estimation data, the outlier removal criteria, and the estimation model.

The generating the estimation model may include: removing outlier data from the calibration data based on the outlier removal criteria; and generating the estimation model based on the calibration data, from which the outlier data is removed.

The setting the outlier removal criteria may include setting the outlier removal criteria for each wavelength of the calibration data set.

The setting the outlier removal criteria may include detecting an outlier for each wavelength from the calibration data set by applying one or more outlier detection techniques, and setting the outlier removal criteria for each wavelength based on the detected outlier.

The estimating the bio-information may include: removing the outlier data from the estimation data based on the outlier removal criteria; and estimating the bio-information based on the estimation data, from which the outlier data is removed, and the estimation model.

The method of estimating bio-information may further include: detecting outlier data from the estimation data based on the outlier removal criteria; and based on the detecting the outlier data, controlling a sensor to re-obtain corresponding estimation data.

The method of estimating bio-information may further include outputting one or more of the outlier removal criteria, the estimation model, and the bio-information.

### BRIEF DESCRIPTION OF THE DRAWINGS

The above and/or other aspects will be more apparent by describing certain example embodiments, with reference to the accompanying drawings, in which:

FIG. 1 is a block diagram illustrating an apparatus for estimating bio-information according to an example embodiment;

FIG. 2 is a block diagram illustrating an apparatus for estimating bio-information according to another example embodiment;

FIGS. 3A and 3B are block diagrams illustrating a processor according to embodiments of the present disclosure;

FIGS. 4A, 4B, 4C, and 4D are diagrams explaining outlier removal criteria;

FIG. 5 is a flowchart illustrating a method of estimating bio-information according to an example embodiment;

FIG. 6 is a flowchart illustrating a method of estimating bio-information according to another example embodiment; and

FIG. 7 is a diagram illustrating a wearable device according to an example embodiment.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail. The relative size and depiction of the elements may be exaggerated for clarity, illustration, and convenience.

It will be understood that, although the terms first, second, etc. may be used herein to describe various elements, these elements should not be limited by these terms. These terms are only used to distinguish one element from another. Also, the singular forms are intended to include the plural forms as well, unless the context clearly indicates otherwise. In the specification, unless explicitly described to the contrary, the word “comprise” and variations, such as “comprise” or “comprising”, will be understood to imply the inclusion of stated elements but not the exclusion of any other elements. Terms, such as ‘part’ and ‘module’ denote units that process at least one function or operation and they may be implemented by using hardware, software, or a combination thereof.

Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

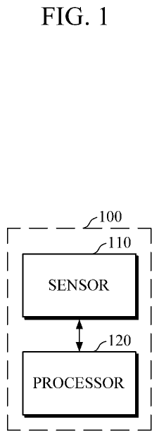

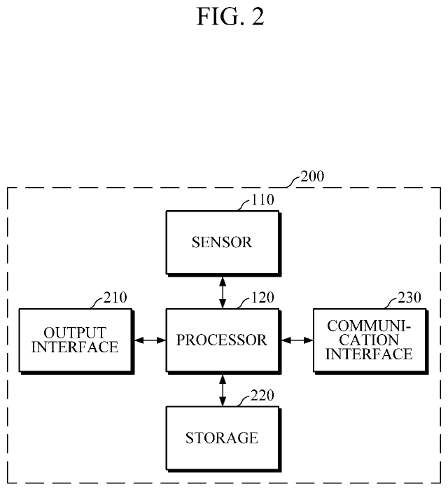

FIG. 1 is a block diagram illustrating an apparatus for estimating bio-information according to an example embodiment. FIG. 2 is a block diagram illustrating an apparatus for estimating bio-information according to another example embodiment.

Referring to FIGS. 1 and 2, the apparatuses 100 and 200 for estimating bio-information according to embodiments of the present disclosure include a sensor 110 and a processor 120.

The sensor 110 may obtain data related to estimating bio-information from an object. The sensor 110 may include a light source for emitting light onto the object, and a detector for detecting scattered or reflected the light when the light, emitted by the light source onto the object, is absorbed into or scattered or reflected from the object. The light source may include a light emitting diode (LED), a laser diode (LD), a phosphor, and the like, but is not limited thereto. The light source may be formed of an array of a plurality of light sources to emit light of a plurality of wavelengths. Each light source may be positioned at different distances from the detector. The detector may include a photo diode, a photo transistor (PTr), an image sensor (e.g., CMOS image sensor), and the like, but is not limited thereto.

The processor 120 may control the sensor 110 at a calibration time to obtain a calibration data set. The processor 120 may control the sensor 110 a plurality of number of times at predetermined time intervals to measure a plurality of calibration data, and may obtain the plurality of calibration data as one calibration data set. Further, each calibration data included in the calibration data set may be discrete multivariate data, e.g., absorbance data at a plurality of predetermined wavelengths rather than spectrum data at continuous wavelengths. In this case, the number of calibration data required for calibration, i.e., the number of data to be included in the calibration data set, may be preset by preprocessing to ensure sufficient reliability.

Once the calibration data set is obtained, the processor 120 may set outlier removal criteria and may generate an estimation model for estimating bio-information.

Further, upon receiving a request for estimating bio-information, the processor 120 may control the sensor 110 to obtain an estimation data set. The processor 120 may control the sensor 110 a plurality of number of times at predetermined time intervals to measure an estimation data set including a plurality of estimation data. Each estimation data included in the estimation data set may include absorbance data at each wavelength. In this case, the number of estimation data required for estimating bio-information, i.e., the number of data to be included in the estimation data set, may be preset by preprocessing to ensure sufficient reliability.

Upon obtaining the estimation data set, the processor 120 may estimate bio-information by using the outlier removal criteria obtained at the calibration time, and the estimation model. In particular, bio-information may include blood glucose, calories, alcohol, triglyceride, protein, cholesterol, uric acid, carotenoid, and the like, but is not limited thereto.

Based on a result of removing an outlier from the estimation data set by using the outlier removal criteria and/or a result of estimating bio-information by using the estimation model, the processor 120 may determine whether to re-perform calibration for a user.

For example, if a percentage of outlier data, detected from the estimation data set, is greater than or equal to a predetermined threshold (e.g., 10%), the processor 120 may determine that calibration is required. Alternatively, if a detection frequency of outlier data at a specific wavelength is relatively higher than other wavelengths, the processor 120 may determine that calibration is required. In addition, if a bio-information estimation result during a predetermined period of time falls outside a predetermined normal range a predetermined number of times or more, the processor 120 may determine that calibration is required. However, this is merely an example, and the processor 120 is not specifically limited thereto and may determine whether it is required to re-perform calibration at predetermined intervals, in response to a user's request, or based on various other criteria.

Referring to FIG. 2, an apparatus 200 for estimating bio-information according to an example embodiment further includes an output interface 210, a storage 220, and a communication interface 230, in addition to the sensor 110 and the processor 120.

The output interface 210 may provide results, processed by the sensor 110 and the processor 120, for a user. For example, the output interface 210 may visually output information, such as the set outlier removal criteria, an estimated bio-information value, and the like, by using a display module. Alternatively, the output interface 210 may output the information in a non-visual manner by voice, vibrations, tactile sensation, and the like, using a speaker module, a haptic module, or the like. In this case, if an estimated blood pressure value falls outside a normal range, the output interface 210 may output warning information in various manners, such as highlighting an abnormal value in red and the like, displaying the abnormal value along with a normal range, outputting a voice warning message, adjusting a vibration intensity, and the like.

The storage 220 may store information, such as the calibration data and the estimation data, and the like which are obtained by the sensor 110. Further, the storage 220 may store information related to driving conditions of the sensor 110, e.g., wavelengths, light intensity, duration, and the like of each light source. In addition, the storage 220 may store information processed by the processor 120, e.g., the outlier removal criteria, the estimation model, the bio-information estimation result, and the like. Moreover, the storage 220 may store a variety of other reference information required for estimating bio-information, e.g., user characteristic information including a user's age, sex, health condition, and the like, but is not limited thereto.

In this case, the storage 220 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The communication interface 230 may communicate with an external device by using wired or wireless communication techniques, to transmit and receive various data to and from the external device. For example, the communication interface 230 may transmit the outlier removal criteria, the estimation model, the bio-information estimation result, and the like to the external device. In this case, examples of the external device may include information processing devices used by ordinary people, such as a smartphone, a tablet PC, a desktop computer, a laptop computer, and the like, as well as medical equipment and the like used in specialized medical institutions.

In this case, examples of the communication techniques may include Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G, 4G, and 5G telecommunications, and the like. However, this is merely exemplary and is not intended to be limiting.

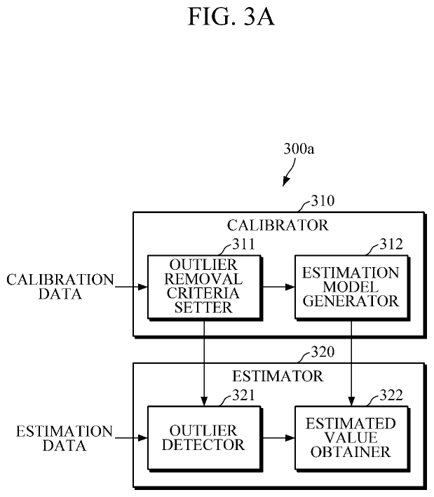

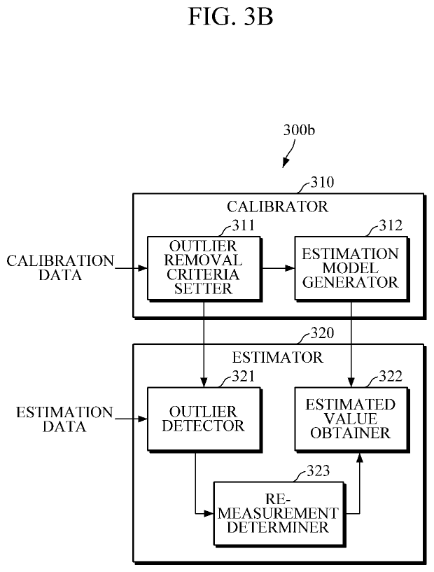

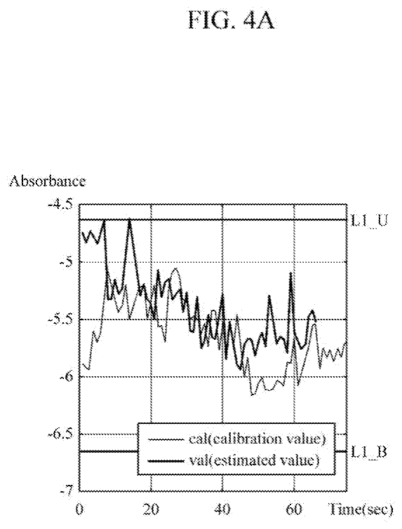

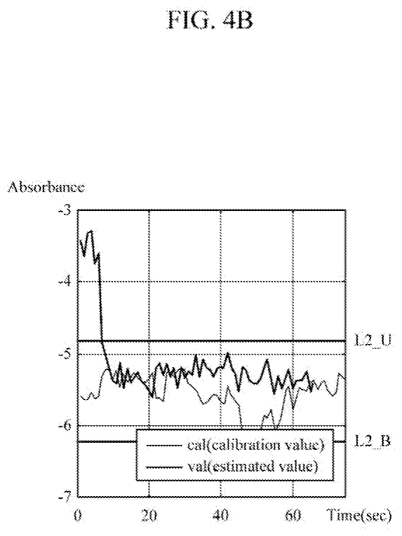

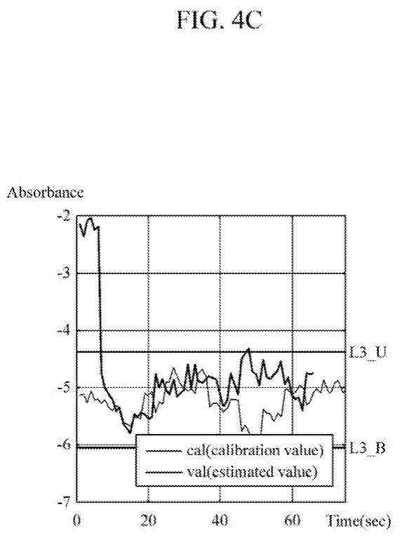

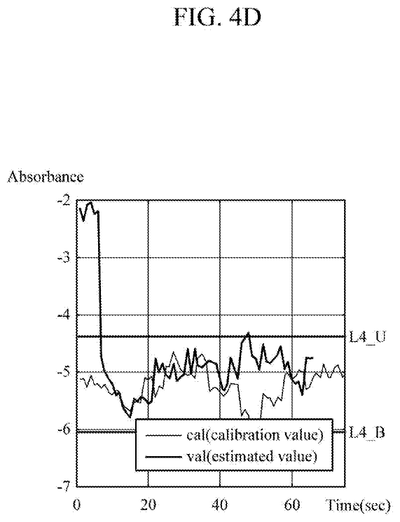

FIGS. 3A and 3B are block diagrams illustrating a processor according to embodiments of the present disclosure. FIGS. 4A to 4D are diagrams explaining outlier removal criteria.

Referring to FIG. 3A, a processor 300a according to an example embodiment includes a calibrator 310 and an estimator 320.

The calibrator 310 includes an outlier removal criteria setter 311 and an estimation model generator 312.

Upon receiving a calibration data set from the sensor 110, the outlier removal criteria setter 311 may set outlier removal criteria for each wavelength by using outlier detection techniques. In this case, the outlier detection techniques may include various known techniques, such as a statistics-based technique, a distance-based technique, a depth-based technique, a density-based technique, and the like.

For example, as an example of the statistics-based technique, a median method is used, which returns “True” for elements more than three scaled median absolute deviations (MAD) away from the median. In this case, the scaled MAD is defined as c*median(abs(A-median(A))), where c=−1/(sqrt(2)*erfcinv(3/2). However, the detection technique is not limited thereto, and various techniques may also be used, including a mean method which returns “True” for elements more than three standard deviations from the mean, a quartile method which returns “True” for elements more than 1.5 interquartile ranges above the upper quartile or below the lower quartile, an extreme studentized deviate test method, and the like.

The outlier removal criteria setter 311 may detect an outlier from the calibration data set by applying any one of various outlier detection techniques, and may set outlier removal criteria based on an outlier detection result. Alternatively, by applying a plurality of outlier detection techniques, the outlier removal criteria setter 311 may detect an outlier from the calibration data set for each outlier detection technique, and may set outlier removal criteria based on the outlier detection result of each detection technique.

The outlier removal criteria setter 311 may set outlier removal criteria for each wavelength. FIGS. 4A to 4D are diagrams illustrating an example of absorbance data at four wavelengths, in which outlier removal criteria, i.e., upper limits L1_U, L2_U, L3_U, and L4_U and lower limits L1_B, L2_B, L3_B, and L4_B of a predetermined normal range, may be set for each wavelength. The outlier removal criteria setter 311 may apply different detection techniques for each wavelength. The outlier removal criteria setter 311 may apply a plurality of detection techniques for each wavelength, and may set outlier removal criteria for each wavelength by using any one of outlier detection results of the detection techniques, or by properly combining the outlier detection results.

For example, upon detecting an outlier by using any one statistical technique for each wavelength of a plurality of calibration data included in the calibration data set, the outlier removal criteria setter 311 may adjust an upper limit and a lower limit so that a percentage of the outlier may fall within a predetermined threshold (e.g., 10%). Alternatively, the outlier removal criteria setter 311 may detect each outlier by applying a plurality of statistical techniques for each wavelength, and may set an upper limit and a lower limit based on statistics, such as an average, of probabilities of occurrence of outliers for each statistical technique. However, this is merely an example, and the outlier removal criteria setter 311 is not limited thereto.

Once the outlier removal criteria are set for each wavelength of the calibration data, the estimation model generator 312 may remove outlier data, in which an outlier is detected, from the calibration data set. In this case, if a specific wavelength of any calibration data in the calibration data set is an outlier, the calibration data may be referred to as outlier data. Further, the estimation model generator 312 may generate an estimation model by using the calibration data set, from which the outlier is removed. For example, the following Equation 1 is an example of the estimation model, but the estimation model is not limited thereto.

Y=a₁X₁+a₂X₂+a₃X₃+a₄X₄ [Equation 1]

Herein, Y denotes an estimated bio-information value to be obtained, and X₁, X₂, X₃, and X₄ denote variable values of each calibration data, i.e., absorbance at four wavelengths. However, this is merely an example, and the number of variables is not specifically limited. In addition, a₁, a₂, a₃, and a₄ denote values obtained by using the calibration data set.

That is, upon removing the outlier data from the calibration data set, the estimation model generator 312 may obtain coefficients a₁, a₂, a₃, and a₄ of each variable by performing regression analysis on the estimation model by using the calibration data, from which the outlier is removed, and reference bio-information obtained at the calibration time, for example, an actual blood glucose value measured by an external blood glucose measuring device.

In addition, based on a result of removing the outlier data from the calibration data set by applying the outlier removal criteria, the estimation model generator 312 may determine reliability of the calibration data set. Upon determination, if reliability of the calibration data set is not suitable for generating an estimation model, the estimation model generator 312 may control the sensor 110 to re-measure calibration data.

For example, based on the result of removing the outlier data, if the number of calibration data in the calibration data set is less than a predetermined threshold, or if a number of times that an outlier is detected for a specific wavelength in the calibration data set is determined to be relatively abnormal compared to other wavelengths, the estimation model generator 312 may determine that calibration data is required to be re-measured. However, this is merely an example, and various other criteria may be set.

The estimator 320 includes an outlier detector 321 and an estimated value obtainer 322.

Upon receiving the estimation data set from the sensor 110, the outlier detector 321 may detect outlier data by applying the outlier removal criteria for each wavelength, which are set by the outlier removal criteria setter 311 at the calibration time.

Upon removing the outlier data, which is detected by the outlier detector 321, from the estimation data set, the estimated value obtainer 322 may obtain an estimated bio-information value for each estimation data by applying the estimation model. Further, the estimated value obtainer 322 may determine, as an estimated bio-information value, statistics of the estimated bio-information values of each estimation data, e.g., mean value, median value, mode, maximum value, minimum value, and the like.

Referring to FIG. 3B, a processor 300b according to an example embodiment includes the calibrator 310 and the estimator 320. Further, the calibrator 310 includes the outlier removal criteria setter 311 and the estimation model generator 312. In addition, the estimator 320 includes the outlier detector 321, the estimated value obtainer 322, and a re-measurement determiner 323. Hereinafter, description will be made based on parts that do not overlap with FIG. 3A.

The re-measurement determiner 323 may determine whether to re-measure data based on an outlier detection result of the outlier detector 321.

For example, the re-measurement determiner 323 may control the sensor 110 to re-measure data every time outlier data is detected from the estimation data set. For example, in the estimation data set including 10 estimation data, if a value of third estimation data at a specific wavelength is detected as an outlier, the re-measurement determiner 323 may determine the third estimation data as outlier data, and may control the sensor 1110 to re-measure the third estimation data. In this case, the re-measurement determiner 323 may drive only a light source of the specific wavelength, at which the outlier is detected, to re-obtain data of the wavelength. Alternatively, the re-measurement determiner 323 may drive all the light sources to re-obtain the third estimation data.

If a predetermined full number of estimation data sets are obtained by re-measurement, the estimated value obtainer 322 may obtain an estimated bio-information value by applying an estimation model. As described above, the estimated value obtainer 322 may obtain estimated values for each estimation data by applying an estimation model, and may determine, as a final estimated bio-information value, statistics of the obtained values.

As described above, in the example embodiment, if the outlier data is detected from the estimation data set for estimating bio-information, re-measurement is performed to obtain normal data, such that an estimation data set with reliability may be obtained, accuracy in estimating bio-information may be improved.

In another example, if re-measurement criteria for the estimation data set are satisfied, the re-measurement determiner 323 may determine to re-measure data. For example, if a percentage of outlier data, detected from the estimation data set, is greater than or equal to a predetermined threshold (e.g., 10%), the re-measurement determiner 323 may control the sensor 110 to re-measure estimation data detected as outlier data.

In this case, if the re-measurement determiner 323 determines not to re-measure the data, the estimated value obtainer 322 may remove the detected outlier data from the estimation data set, and may estimate bio-information by using the remaining estimation data.

Generally, if re-measurement of data or re-estimation of bio-information is performed by generating an estimation model at the calibration time without separately removing an outlier and then verifying the estimation model, or by estimating bio-information at a bio-information estimation time by using an estimation model, from which an outlier is not removed, and then verifying a bio-information estimation result, time delay of bio-information estimation may be increased, or accuracy in estimating bio-information may be reduced.

By contrast, in the embodiments described above, by obtaining outlier removal criteria, which are personalized for each user based on the calibration data set obtained for each user at the calibration time, and generating an estimation model, an outlier may be detected and may be removed directly from the estimation data set for estimating bio-information, or data may be re-measured, such that reliability of the estimation data set may be increased, and accuracy of an estimated bio-information values may be improved.

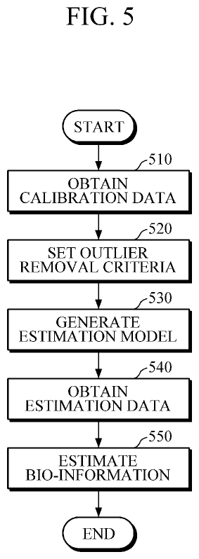

FIG. 5 is a flowchart illustrating a method of estimating bio-information according to an example embodiment.

FIG. 5 may be an example of a method of estimating bio-information which is performed by the apparatuses 100 and 200 for estimating bio-information according to the embodiments of FIGS. 1 and 2.

The apparatuses 100 and 200 for estimating bio-information may obtain a calibration data set, including a plurality of calibration data, from a user at a calibration time in operation 510. Each calibration data may be discrete multivariate data including absorbance at a plurality of wavelengths. The apparatuses 100 and 200 for estimating bio-information may obtain a plurality of calibration data by driving a sensor at predetermined time intervals. In this case, the sensor may include a plurality of light sources for emitting light of a plurality of wavelengths to obtain multivariate data, and a detector for detecting reflected or scattered light when light, emitted by the light sources onto an object, is reflected or scattered from the object.

Then, the apparatuses 100 and 200 for estimating bio-information may set outlier removal criteria in operation 520 by using the calibration data set obtained in operation 510. For example, the apparatuses 100 and 200 for estimating bio-information may detect an outlier by applying an outlier detection technique for each wavelength, and may set an upper limit and a lower limit of a normal absorbance range for each wavelength as the outlier removal criteria. For example, the apparatuses 100 and 200 for estimating bio-information may apply any one outlier detection technique for each wavelength or a plurality of outlier detection techniques for each wavelength, and may set the outlier removal criteria by using any one outlier detection result of each detection technique or by properly combining outlier detection results of two or more outlier detection techniques.

Subsequently, the apparatuses 100 and 200 for estimating bio-information may remove the detected outlier data from the calibration data set, and may generate an estimation model by using the calibration data set, from which the outlier data is removed, in operation 530. As described above, the estimation model may be defined as linear combination using multivariate values as input, i.e., absorbance at each wavelength as input. However, the estimation model is not limited thereto. As described above, the apparatuses 100 and 200 for estimating bio-information may generate an estimation model by obtaining coefficients of each variable by inputting absorbance at each wavelength of normal calibration data, from which the outlier data is removed, into an estimation model.

Next, upon receiving a request for estimating bio-information, the apparatuses 100 and 200 for estimating bio-information may obtain an estimation data set by using the sensor in operation 540. The request for estimating bio-information may be input by a user, or may be received at predetermined intervals or from an external device. The apparatuses 100 and 200 for estimating bio-information may control the sensor at predetermined time intervals to obtain a predetermined number of estimation data.

Then, the apparatuses 100 and 200 for estimating bio-information may estimate bio-information in operation 550 by using the outlier removal criteria set in operation 520, the estimation model generated in operation 530, and the estimation data set obtained in operation 540. The apparatuses 100 and 200 for estimating bio-information may detect outlier data from the estimation data set by using the outlier removal criteria, and may remove the outlier data from the estimation data set. Further, by inputting the remaining estimation data, from which the outlier data is removed, into the estimation model, the apparatuses 100 and 200 for estimating bio-information may obtain estimated bio-information values for each estimation data, and may determine statistics of the obtained estimated values as an estimated bio-information value.

Upon obtaining the estimated bio-information value, the apparatuses 100 and 200 for estimating bio-information may provide the obtained estimated bio-information value for a user. For example, the apparatuses 100 and 200 for estimating bio-information may visually output information, such as the estimated bio-information value, a bio-information estimation history, a user's health condition determined based on the estimated bio-information value, and the like, on a display. Further, the apparatuses 100 and 200 for estimating bio-information may output the information in a non-visual manner by voice, vibrations, tactile sensation, and the like, using a speaker, a haptic device, or the like.

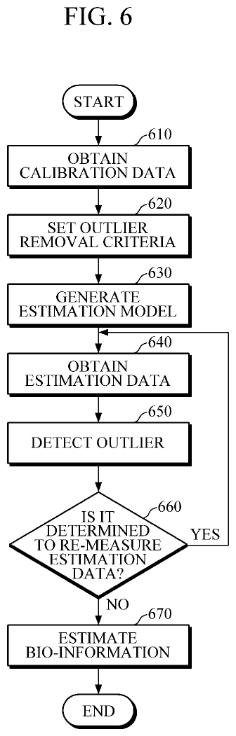

FIG. 6 is a flowchart illustrating a method of estimating bio-information according to another example embodiment.

FIG. 6 may be an example of a method of estimating bio-information which is performed by the apparatuses 100 and 200 for estimating bio-information according to the embodiments of FIGS. 1 and 2.

The apparatuses 100 and 200 for estimating bio-information may obtain a calibration data set, including a plurality of calibration data, from a user at a calibration time in operation 610. The apparatuses 100 and 200 for estimating bio-information may obtain a plurality of calibration data by driving a sensor at predetermined time intervals.

Ten, the apparatuses 100 and 200 for estimating bio-information may set outlier removal criteria in operation 620 by using the calibration data set obtained in operation 610. For example, the apparatuses 100 and 200 for estimating bio-information may detect an outlier by applying various outlier detection techniques. Further, upon detecting the outlier by using outlier detection techniques, the apparatuses 100 and 200 for estimating bio-information may set the outlier removal criteria for each wavelength based on an outlier detection result.

Subsequently, the apparatuses 100 and 200 for estimating bio-information may remove the detected outlier data from the calibration data set, and may generate an estimation model by using the calibration data set, from which the outlier data is removed in operation 630.

Next, upon receiving a request for estimating bio-information, the apparatuses 100 and 200 for estimating bio-information may obtain an estimation data set by using a sensor in operation 640. The number of the estimation data included in the estimation data set may be preset properly by preprocessing, so as to improve reliability of an estimated bio-information value.

Then, the apparatuses 100 and 200 for estimating bio-information may detect an outlier in operation 650 from the estimation data set, obtained in operation 640, by using the outlier removal criteria set in operation 620. Upon detecting a specific wavelength of specific estimation data as an outlier, the apparatuses 100 and 200 for estimating bio-information may determine the estimation data as outlier data.

Upon detecting the outlier in operation 650, the apparatuses 100 and 200 for estimating bio-information may determine to re-measure the estimation data, in which the outlier is detected, in operation 660. For example, in order to obtain a predetermined full number of estimation data, the apparatuses 100 and 200 for estimating bio-information may determine to re-measure the estimation data every time the outlier data is detected from the estimation data set. In another example, if the number of estimation data, which are detected as outlier data, in the entire estimation data set is greater than a predetermined percentage of a total number of the entire estimation data, the apparatuses 100 and 200 for estimating bio-information may determine to re-measure the estimation data.

Upon determining to re-measure the estimation data, the apparatuses 100 and 200 for estimating bio-information may return to operation 640 to re-obtain the estimation data. The operations 640 to 660 may be repeated until a predetermined number of estimation data are obtained, so as to ensure reliability of an estimated bio-information value.

Subsequently, upon obtaining the predetermined number of estimation data in the operations 640 to 660, the apparatuses 100 and 200 for estimating bio-information may determine estimated bio-information values based on each estimation data by applying the estimation model generated in operation 630, and may obtain statistics of the determined plurality of estimated bio-information values as an estimated bio-information value in operation 670.

Next, the apparatuses 100 and 200 for estimating bio-information may output the bio-information estimation result in various visual/non-visual manners and may provide the estimation result for a user.

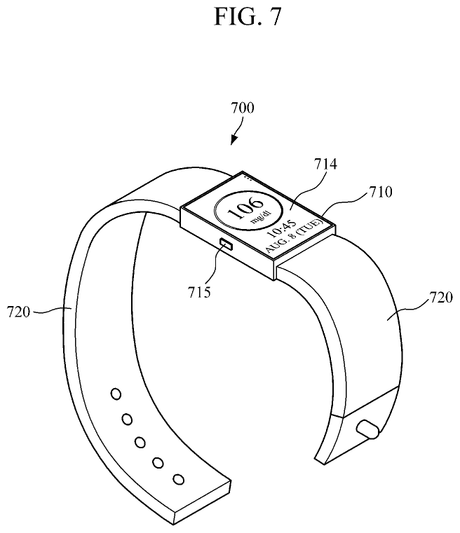

FIG. 7 is a diagram illustrating a wearable device according to an example embodiment. Various embodiments of the apparatuses 100 and 200 for estimating bio-information described above may be mounted in a smart watch-type wearable device worn on a user's wrist, as illustrated in FIG. 7. However, the wearable device is not limited thereto, and may be wearable devices of various types, such as a bracelet-type wearable device, a wristband-type wearable device, a ring-type wearable device, a glasses-type wearable device, a headband-type wearable device, and the like. Further, in addition to a wearable device worn on an object, the wearable device may be mounted in a mobile device, such as a smartphone, a tablet PC, and the like, which may be carried by a user.

Referring to FIG. 7, the wearable device 700 includes a main body 710 and a strap 720.

Various modules of various embodiments of the aforementioned apparatuses 100 and 200 for estimating bio-information may be mounted inside or outside of the main body 710. Further, modules for performing other functions, e.g., time, alarm, messenger, etc., may be mounted in the main body 710.

The strap 720 may be connected to the main body 710. The strap 720 may be flexible so as to be wrapped around a user's wrist. The strap 720 may be bent in a manner that allows the strap 720 to be detached from the user's wrist or may be formed as a band that is not detachable. Air may be injected into the strap 720 or an airbag may be included in the strap 720, so that the strap 720 may have elasticity according to a change in pressure applied to the wrist, and the change in pressure of the wrist may be transmitted to the main body 710.

A battery may be embedded in the main body 710 or the strap 720 to supply power to the wearable device 700.

The wearable device 700 may include a sensor which is mounted in the main body 710 to obtain discrete multivariate data from a user's wrist. The sensor may include a light source, and a detector. The light source may be mounted at the bottom of the main body 710 to be exposed to a user's wrist, so that the light source may emit light onto the wrist. The light source may be formed of an array of a plurality of LEDs to emit light of a plurality of wavelengths. However, the light source is not limited thereto, and may be formed of a laser diode and the like. The detector may include a photo diode, and may detect light returning from the user's skin, may convert a light signal into an electric signal, and may output the signal.

The processor may receive a user's instruction input through a manipulator 715 or a display 714, and may process required operations according to the received instruction. For example, the processor may be electrically connected with the sensor, and may control the sensor upon receiving an instruction for calibration or estimating bio-information.

Upon receiving a request for calibration, the processor may control the sensor a plurality of number of times at predetermined time intervals to obtain a calibration data set including a plurality of calibration data. Upon obtaining the calibration data set, the processor may set outlier removal criteria by using the obtained calibration data set. For example, by applying one or more outlier detection techniques for each wavelength, the processor may detect an outlier for each detection technique, and may set outlier removal criteria for each wavelength by using an outlier detection result.

Upon setting the outlier removal criteria, the processor may store the outlier removal criteria in a storage mounted in the main body 710. Further, the processor may remove outlier data from the calibration data set according to the set outlier removal criteria, and may generate an estimation model for estimating bio-information by using the calibration data set, from which the outlier data is removed.

In addition, upon receiving an instruction for estimating bio-information, the processor may control the sensor to obtain an estimation data set. Furthermore, by referring to the outlier removal criteria stored in the storage, the processor may detect an outlier from the estimation data set.

In one embodiment, upon detecting the outlier, the processor may remove outlier data from the estimation data set, and may obtain an estimated bio-information value by using the remaining estimation data and the estimation model.

In another embodiment, upon detecting the outlier, the processor may control the sensor to re-measure the estimation data, and once a predetermined number of estimation data are obtained, the processor may obtain an estimated bio-information value by applying the estimation model.

A display 714 may be mounted on a front surface of the main body 710. The display 714 may include a touch screen for receiving a user's touch input. The display 714 may receive the touch input and transmit the touch input to the processor, and may output a processing result of the processor. For example, the display 174 may display a bio-information estimation result, and may display additional information, such as a bio-information estimation history, a change in health condition, warning, and the like, along with the estimation result.

Further, the main body 710 may also include a manipulator 715 which receives a user's instruction and transmits the received instruction to the processor. The manipulator 715 may include a power button to input a command to turn on/off the wearable device 700.

Moreover, a communication interface for communication with an external device may be mounted in the main body 710. The communication interface may output a bio-information estimation result through an external device, e.g., an output module of a user's mobile device, or may transmit the estimation result to an external device so that the result may be stored in a storage module of the external device. Further, the communication interface may receive information for supporting various other functions, performed by the external device, and the like from the external device.

In addition, the main body 710 may include a temperature sensor for measuring temperature on a user's wrist and a humidity sensor for measuring humidity on the wrist, and the like. The processor, mounted in the main body 710, may monitor a measurement environment by using temperature/humidity information obtained by the temperature sensor/the humidity sensor, and the like, and may provide guide information on measurement environment for a user, or may use the information for correcting an estimated bio-information value.

While not restricted thereto, one or more example embodiments can be embodied as computer-readable code on a computer-readable recording medium. The computer-readable recording medium is any data storage device that can store data that can be thereafter read by a computer system. Examples of the computer-readable recording medium include read-only memory (ROM), random-access memory (RAM), CD-ROMs, magnetic tapes, floppy disks, and optical data storage devices. The computer-readable recording medium can also be distributed over network-coupled computer systems so that the computer-readable code is stored and executed in a distributed fashion. Also, an example embodiment may be written as a computer program transmitted over a computer-readable transmission medium, such as a carrier wave, and received and implemented in general-use or special-purpose digital computers that execute the programs. Moreover, it is understood that in example embodiments, one or more units of the above-described apparatuses and devices can include circuitry, a processor, a microprocessor, etc., and may execute a computer program stored in a computer-readable medium.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for estimating bio-information, the apparatus comprising:
a sensor configured to obtain a calibration data set and an estimation data set from an object; and
a processor configured to set outlier removal criteria and generate an estimation model based on the calibration data set, and to estimate bio-information based on the estimation data set, the outlier removal criteria, and the estimation model.

2. The apparatus of claim 1, wherein the sensor comprises:
at least one light source configured to emit light of a plurality of wavelengths onto the object; and
a detector configured to detect the light when the light is reflected or scattered from the object.

3. The apparatus of claim 1, wherein the processor is further configured to remove outlier data from the calibration data set based on the outlier removal criteria, and to generate the estimation model based on the calibration data set, from which the outlier data is removed.

4. The apparatus of claim 1, wherein the processor is further configured to set the outlier removal criteria for each wavelength of the calibration data set.

5. The apparatus of claim 4, wherein the processor is further configured to detect an outlier for each wavelength from the calibration data set by applying a pre-defined outlier detection technique, and to set the outlier removal criteria for each wavelength based on the detected outlier.

6. The apparatus of claim 1, wherein the processor is further configured to remove outlier data from the estimation data set based on the outlier removal criteria, and to estimate the bio-information based on the estimation data set, from which the outlier data is removed, and the estimation model.

7. The apparatus of claim 1, wherein the processor is further configured to detect outlier data from the estimation data set based on the outlier removal criteria, and upon detecting the outlier data, to control the sensor to re-obtain corresponding estimation data.

8. The apparatus of claim 7, wherein by driving a light source of a wavelength, at which the outlier data is detected, the sensor re-obtains data for the wavelength.

9. The apparatus of claim 7, wherein the sensor comprises a plurality of light sources configured to emit light of a plurality of wavelengths to the object, and the processor is further configured to drive all of the plurality of light sources to re-obtain estimation data for all of the plurality of wavelengths.

10. The apparatus of claim 1, wherein the processor is further configured to detect an outlier from the estimation data set, and determine whether to perform calibration based on the detected outlier.

11. The apparatus of claim 1, further comprising an output interface configured to output at least one of the outlier removal criteria, the estimation model, and the bio-information.

12. The apparatus of claim 1, further comprising a communication interface configured to transmit at least one of the outlier removal criteria, the estimation model, and the bio-information to an external device.

13. The apparatus of claim 1, wherein the bio-information comprises at least one of blood glucose, cholesterol, triglyceride, protein, and uric acid.

14. A method of estimating bio-information, the method comprising:
obtaining calibration data;
setting outlier removal criteria based on the calibration data;
generating an estimation model based on the calibration data;
obtaining estimation data; and
estimating bio-information based on the estimation data, the outlier removal criteria, and the estimation model.

15. The method of claim 14, wherein the generating the estimation model comprises:
removing outlier data from the calibration data based on the outlier removal criteria; and
generating the estimation model based on the calibration data, from which the outlier data is removed.

16. The method of claim 14, wherein the setting the outlier removal criteria comprises setting the outlier removal criteria for each wavelength of the calibration data.

17. The method of claim 14, wherein the setting the outlier removal criteria comprises detecting an outlier for each wavelength from the calibration data by applying one or more outlier detection techniques, and setting the outlier removal criteria for each wavelength based on the detected outlier.

18. The method of claim 14, wherein the estimating the bio-information comprises:
removing outlier data from the estimation data based on the outlier removal criteria; and
estimating the bio-information based on the estimation data, from which the outlier data is removed, and the estimation model.

19. The method of claim 14, further comprising:
detecting outlier data from the estimation data based on the outlier removal criteria; and
based on the detecting the outlier data, controlling a sensor to re-obtain corresponding estimation data.

20. The method of claim 14, further comprising outputting one or more of the outlier removal criteria, the estimation model, and the bio-information.
